(19) United States
(12) Patent Application Publication
(10) Pub. No.: US 2021/0378650 A1
(43) Pub. Date: Dec. 9, 2021

(54) BODY-CAVITY SHEET APPLYING DEVICE AND SHEET APPLYING METHOD

(71) Applicant: Mochida Pharmaceutical Co., Ltd., Tokyo (JP)

(72) Inventors: Hitoshi MIZUNO, Tokyo (JP); Daichi TANAKA, Tokyo (JP)

(73) Assignee: Mochida Pharmaceutical Co., Ltd., Tokyo, (JP)

(21) Appl. No.: 17/293,229

(22) PCT Filed: Nov. 18, 2019
(86) PCT No.: PCT/JP2019/045028

§ 371 Date: May 12, 2021

(30) Foreign Application Priority Data
Nov. 19, 2018 (JP) 2018-216516

(51) Int. Cl. A61B 17/00

(52) U.S. Cl. CPC A61B 17/00491

(57) ABSTRACT

A sheet applying device, a sheet applying system, and a sheet applying method are provided. A first sheet applying system 10 mainly includes a first sheet applying device 11 and a sheet cartridge 900. The first sheet applying device 11 mainly includes a first supporter 200 and a holder 500. The first supporter 200 mainly includes a first support shaft 210a, a second support shaft 210b, a first swing mechanism 220, a support cover 211, and a handle 300. The first support shaft 210a and the second support shaft 210b have straight cylindrical bar shapes and movably extend in parallel to each other from a proximal end side of the first sheet applying system 10 toward a distal end side of the first sheet applying system 10, in other words, in a longitudinal direction of the first supporter 200.

### TECHNICAL FIELD

The present invention relates to a device, a sheet applying system, and a sheet applying method that are used to apply a sheet to a site inside a body.

### DESCRIPTION OF THE RELATED ART

Conventionally, an endoscope hemostatic treatment instrument configured to insert a roll-shaped protection sheet into a body cavity and apply the sheet to a target has been known. Two linear members are provided at a leading end of the endoscope hemostatic treatment instrument. One side of a protection sheet for stopping bleeding at a target inside the body is wound in a roll shape around one of the linear members, and another side of the protection sheet is fixed to the other linear member. When the other linear member is pulled toward a proximal end side after the leading end of the endoscope hemostatic treatment instrument is inserted into the body and the protection sheet is expanded, the protection sheet is separated from the other linear member and applied to the target (refer to Patent Literature 1).

### RELATED ART LIST

### Patent Literature

Patent Literature 1: Japanese Patent Laid-Open No. 2012-40135

### SUMMARY OF THE INVENTION

### Technical Problem

However, with the configuration in which the linear members are pulled toward the proximal end side so that the protection sheet is separated and then applied, the protection sheet potentially moves and cannot be applied to a desired position. Furthermore, when the linear members are pulled toward the proximal end side while the protection sheet is being applied to the target, the endoscope needs to be moved in a direction opposite to the direction in which the linear members are pulled so that a site in contact with the protection sheet is prevented from being displaced relative to the target.

According to the present invention, a sheet applying device, a sheet applying system, and a sheet applying method intended to solve such a problem are provided.

### Solution to the Problem

The present application invention includes [1] to [24] below.

[Invention 1]

A sheet applying device comprising:

a supporter configured to be inserted into a tube provided through a body wall; and

a holder configured to be provided at a leading end of the supporter and capable of holding a sheet, and to deform to eject the sheet while expanding the sheet, after the holder holding the sheet protrudes into a body cavity through a leading end of the tube.

[Invention 2]

The sheet applying device according to Invention 1, wherein the holder can apply the sheet to a target while expanding the sheet.

[Invention 3]

The sheet applying device according to Invention 2, wherein the holder can apply the sheet while expanding the sheet after contacting a substantially central part of the sheet to a target.

[Invention 4]

The sheet applying device according to any one of Inventions 1 to 3, wherein

the holder has such a thin plate shape that the holder can be wound around the holder, is pressed in an expanding direction when being wound, and holds the sheet being wound and enclosed on an inner periphery side of the holder, and

after the holder holding the sheet protrudes into the body cavity through the leading end of the tube, the holder expands the sheet and applies the sheet to a target.

[Invention 5]

The sheet applying device according to any one of Inventions 1 to 3, wherein

the supporter includes
a support shaft extending and movable in a longitudinal direction of the supporter, and
a handle configured to move the support shaft, the holder includes
a first opening-closing member and a second opening-closing member that are attached to a distal end of the support shaft, and
a first holding end part and a second holding end part that are attached to distal ends of the first and second opening-closing members, respectively, and

when the support shaft is moved toward a distal end by the handle, the first and second opening-closing members are separated from each other due to pressing by the support shaft and the first and second holding end parts are separated from each other.

[Invention 6]

The sheet applying device according to Invention 5, wherein

the support shaft includes a first support shaft and a second support shaft extending in parallel to each other,

the handle is attached to proximal ends of the first and second support shafts and moves the first support shaft and/or the second support shaft in the longitudinal direction of the supporter,

the first and second opening-closing members are attached to distal ends of the first and second support shafts, respectively, and

when the first and second support shafts are moved toward the distal ends, the first and second opening-closing members are separated from each other due to pressing by the first and second support shafts and the first and second holding end parts are separated from each other.

[Invention 7]

The sheet applying device according to Inventions 5 or 6, further comprising:

a holding-movement part attached to proximal ends of the first and second opening-closing members;

a first holding operation part rotatably attached to the first opening-closing member;

a second holding operation part rotatably attached to the second opening-closing member;

a first holding support part rotatably attached to the first holding operation part; and

a second holding support part rotatably attached to the second holding operation part, wherein

the first holding operation part has a distal end attached to the first opening-closing member and has a proximal end attached to the holding-movement part,

the second holding operation part has a distal end attached to the second opening-closing member and has a proximal end attached to the holding-movement part,

the holding-movement part includes a long hole on a distal end side,

the proximal ends of the first and second holding operation parts are rotatably attached to the holding-movement part, and

the distal ends of the first and second holding support parts are movably attached in the long hole.

[Invention 8]

The sheet applying device according to any one of Inventions 5 to 7, wherein the first and second holding end parts can be separated in parallel to each other.

[Invention 9]

The sheet applying device according to any one of Inventions 6 to 8, wherein

the handle moves the first support shaft in the longitudinal direction of the supporter, and

when the first support shaft is moved toward the distal end, the first opening-closing member is pressed by the first support shaft and the first and second opening-closing members swing in a plane including the first and second support shafts.

[Invention 10]

The sheet applying device according to any one of Inventions 1 to 3, wherein

the supporter includes a cylindrical part and a pusher stored at a distal end in the cylindrical part, and

the pusher can protrude and expand from the cylindrical part to eject the sheet while expanding the sheet.

[Invention 11]

The sheet applying device according to any one of Inventions 1 to 3, wherein

the supporter includes a bar part,

the holder includes a bending part provided at a leading end of the bar part, and

the bending part can bend to eject the sheet while expanding the sheet.

[Invention 12]

The sheet applying device according to any one of Inventions 1 to 11, wherein the opening-closing member includes a connection part connectable with a surgical operation support robot.

[Invention 13]

A sheet cartridge used for the sheet applying device according to any one of Inventions 1 to 3, the sheet cartridge comprising:

a first shaft and a second shaft having bar shapes;

a sheet including a first part and a second part, the first part being wound around the first shaft, the second part being different from the first part and wound around the second shaft; and

a cover attached to cover at least part of an outer surface of the sheet wound around the first and second shafts.

[Invention 14]

The sheet cartridge according to Invention 13, wherein the cover includes a string extending from a proximal end of the cover.

[Invention 15]

A sheet applying system comprising:

the sheet applying device according to any one of Inventions 1 to 12; and

the sheet cartridge according to Invention 13 or 14.

[Invention 16]

A sheet applying method of using the sheet applying device according to any one of Inventions 1 to 3, the sheet applying method comprising:

a step of protruding at least part of the sheet into the body cavity from the leading end of the tube through inside of the tube;

a step of expanding at least part of the sheet;

a step of causing at least part of the sheet to contact a target; and

a step of entirely expanding the sheet so that the sheet comes off the first and second shafts.

[Invention 17]

The sheet applying method according to Invention 16, wherein the coming-off step includes a step of applying the sheet to a target.

[Invention 18]

The sheet applying method according to Invention 16, wherein

the contacting step includes a step of causing a substantially central part of the sheet to contact a target, and

the coming-off step is executed after the contacting step and includes a step of applying the sheet to a target.

[Invention 19]

The sheet applying method according to any one of Inventions 16 to 18, wherein the steps are executed

in an order of the protruding step, the expanding step, the contacting step, the coming-off step, and the applying step, or

in an order of the protruding step, the contacting step, the expanding step, the coming-off step, and the applying step.

[Invention 20]

A sheet applying method of using the sheet applying system according to Invention 15, the sheet applying method comprising:

a step of protruding at least part of the sheet into the body cavity from the leading end of the tube through inside of the tube;

a step of expanding at least part of the sheet;

a step of causing at least part of the sheet to contact a target; and

a step of entirely expanding the sheet so that the sheet comes off the first and second shafts.

[Invention 21]

The sheet applying method according to Invention 20, wherein the coming-off step includes a step of applying the sheet to a target.

[Invention 22]

The sheet applying method according to Invention 20, wherein

the contacting step includes a step of causing a substantially central part of the sheet to contact a target, and

the coming-off step is executed after the contacting step and includes a step of applying the sheet to a target.

[Invention 23]

The sheet applying method according to any one of Inventions 20 to 22, wherein the steps are executed

in an order of the protruding step, the expanding step, the contacting step, the coming-off step, and the applying step, or

in an order of the protruding step, the contacting step, the expanding step, the coming-off step, and the applying step.

[Invention 24]

The sheet applying method according to any one of Inventions 20 to 23, wherein

the cover includes a string extending from a proximal end of the cover, and

the sheet applying method further includes, before the step of expanding at least part of the sheet, a step of removing the cover from the sheet by pulling the string to a proximal end side.

### Effects of the Invention

According to the present invention, a sheet applying device, a sheet applying system, and a sheet applying method intended to solve the above-described problem are provided.

### BRIEF DESCRIPTION OF THE DRAWINGS

FIG. 1 is a front view of a first sheet applying system.

FIG. 2 is a plan view of the first sheet applying system.

FIG. 3 is a partially transparent plan view and a partial front view of the first sheet applying system.

FIG. 4 is a partially transparent plan view, a cross-sectional view, and a partial front view of the first sheet applying system.

FIG. 5 is a partially transparent plan view of a first sheet applying device.

FIG. 6 is a partially transparent plan view of the first sheet applying device.

FIG. 7 is a diagram illustrating the procedure of removing a cover from the first sheet applying system.

FIG. 8 is a diagram illustrating the procedure of applying a sheet to an affected part by using the first sheet applying system.

FIG. 9 is a front view of a second sheet applying system.

FIG. 10 is a partially transparent plan view and a partial front view of the second sheet applying system.

FIG. 11 is a partially transparent plan view and a partial front view of the second sheet applying system.

FIG. 12 is a partially transparent front view of a third sheet applying system.

FIG. 13 is a partially transparent plan view of the third sheet applying system.

FIG. 14 is a partially transparent plan view of the third sheet applying system.

FIG. 15 is a schematic diagram illustrating an endoscope device using the third sheet applying system.

FIG. 16 is a partially transparent front view of a fourth sheet applying system.

FIG. 17 is a partially transparent front view of the fourth sheet applying system.

FIG. 18 is a diagram illustrating the procedure of applying a sheet to an affected part by using the fourth sheet applying system.

FIG. 19 is a diagram illustrating the procedure of applying a sheet to an affected part by using a fifth sheet applying system.

FIG. 20 is a diagram illustrating the procedure of applying a sheet to an affected part by using a sixth sheet applying system.

FIG. 21 is a plan view of a seventh sheet applying system.

FIG. 22 is a plan view of the seventh sheet applying system.

FIG. 23 is a partially transparent plan view, a cross-sectional view, and a partial front view of an eighth sheet applying system.

### REFERENCE NUMERALS LIST

1 body wall

2 target

3 flexible endoscope device

4 forceps opening

10 first sheet applying system

11 first sheet applying device

20 second sheet applying system

21 second sheet applying device

30 third sheet applying system

31 third sheet applying device

40 fourth sheet applying system

41 fourth sheet applying device

200 first supporter

210a first support shaft

210b second support shaft

211 support cover

211a cutout

211b cutout

220 first swing mechanism

221a first swing-support part

221b second swing-support part

222a first swing operation part

222b second swing operation part

223 swing movement part

224 pin

300 handle

301 pin

310 first operation lever

320 second operation lever

400 second supporter

410 support shaft

500 holder

520 first holding end part

540 second holding end part

541 connection hole

544 second holding operation part

554 holding-movement part

555 pin

600 third supporter

610 support wire

700 handle

710 operation part

720 grasping part

810 fourth supporter

811 knob

812 bar part

820 protector

830 holder

831 holding part

832 tongue part

900 sheet cartridge

910 sheet

920 cover

921 string

### DESCRIPTION OF THE PREFERRED EMBODIMENTS

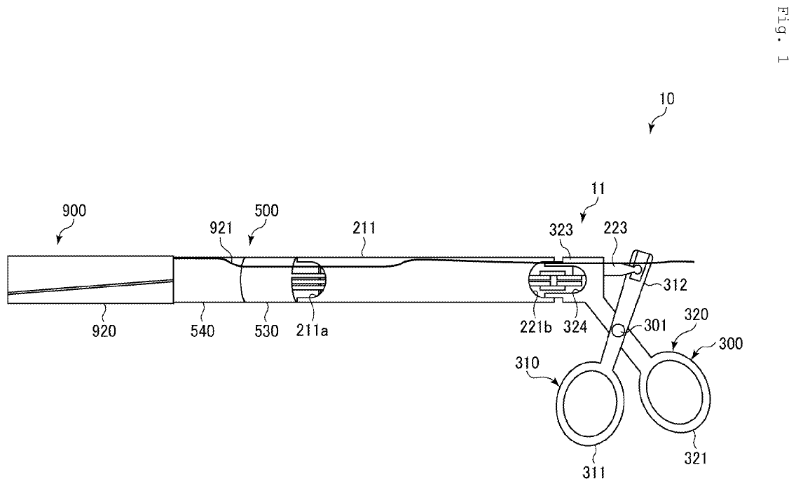

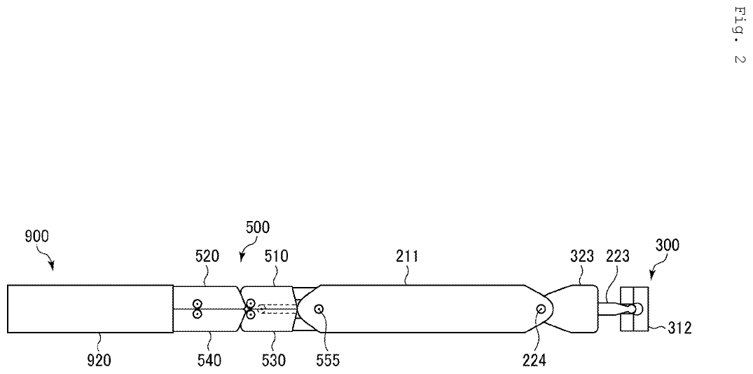

A first sheet applying system 10 according to a first embodiment of the present invention will be described below with reference to FIGS. 1 to 8. Note that, in the present specification, an end part positioned in the direction of insertion into a body cavity is referred to as a distal end, and a site positioned on an operator side is referred to as a proximal end.

The first sheet applying system 10 mainly includes a first sheet applying device 11 and a sheet cartridge 900.

The first sheet applying device 11 mainly includes a first supporter 200 and a first holder 500. The first supporter 200 mainly includes a first support shaft 210a, a second support shaft 210b, a first swing mechanism 220, a support cover 211, and a handle 300. The first support shaft 210a and the second support shaft 210b have straight cylindrical bar shapes and movably extend in parallel to each other from the proximal end side of the first sheet applying system 10 toward the distal end side thereof, in other words, in the longitudinal direction of the first supporter 200.

The first swing mechanism 220 mainly includes a first swing-support part 221a and a second swing-support part 221b having substantially triangular thin-plate shapes, a first swing operation part 222a and a second swing operation part 222b having bar plate shapes, and a swing movement part 223 having a straight bar shape. The first swing-support part 221a and the second swing-support part 221b are pivotally supported by a pin 224 at their corners so that these components are relatively rotatable. Corners positioned on the distal end side among the other corners of the first swing-support part 221a and the second swing-support part 221b are pivotally supported to the first support shaft 210a and the second support shaft 210b so that these components are relatively rotatable. Corners positioned on the proximal end side among the other corners of the first swing-support part 221a and the second swing-support part 221b are pivotally supported to the distal ends of the first swing operation part 222a and the second swing operation part 222b so that these components are relatively rotatable. The first swing operation part 222a and the second swing operation part 222b are pivotally supported at their proximal ends so that the swing operation parts are rotatable relative to the distal end of the swing movement part 223. A proximal end 223a of the swing movement part 223 has a ball shape and is connected with the handle 300.

The support cover 211 is a cylinder extending across substantially the entire lengths of the first support shaft 210a and the second support shaft 210b and has cutouts 211a and 211b having front-view semicircular shapes on the distal and proximal end sides. The proximal end of the support cover 211 is pivotally supported together with the first swing-support part 221a and the second swing-support part 221b by the pin 224 so that these components are relatively rotatable.

The handle 300 mainly includes a first operation lever 310 and a second operation lever 320. The first operation lever 310 mainly includes a first annular part 311 provided with a finger hole, and a first operation bar part 312 extending straight from the first annular part 311. A bottomed cylinder hole 313 having a depth in the longitudinal direction of the first operation bar part 312 is provided at a leading end of the first operation bar part 312. The second operation lever 320 mainly includes a second annular part 321 provided with a finger hole, a second operation bar part 322 extending straight from the second annular part 321 and having a leading end bent at a predetermined angle, and an operation cover 323 having a cylindrical shape.

A through-hole 322a through which the swing movement part 223 movably penetrates is provided at a leading end of the second operation bar part 322, and the operation cover 323 is attached to the leading end. The operation cover 323 extends over the pin 224 from the second operation lever 320 above-described has a cutout 324 having a semicircular shape on the proximal end side. The distal end side of the operation cover 323 is pivotally supported together with the first swing-support part 221a, the second swing-support part 221b, and the proximal end of the support cover 211 by the pin 224 so that these components are relatively rotatable.

The first operation lever 310 and the second operation lever 320 are pivotally supported to be relatively swingable by a pin 301.

The ball-shaped proximal end 223a of the swing movement part 223 is inserted into the bottomed cylinder hole 313. Accordingly, when an operator grips the first operation lever 310 and the second operation lever 320, the proximal end 223a is freely movable in an axial direction in the bottomed cylinder hole 313, and the swing movement part 223 is movable by pressing toward the distal end side by the first operation lever 310.

With the above-described configuration, the handle 300 is attached to the proximal ends of the first support shaft 210a and the second support shaft 210b.

The first holder 500 mainly includes a first opening-closing member 510, a second opening-closing member 530, a first holding end part 520, a second holding end part 540, and a first holding operation part 524, a second holding operation part 544, and a holding-movement part 554 having thin-plate bar shapes.

The first opening-closing member 510 mainly includes a first opening-closing main part 511 having a semicylindrical shape and a semicircular section at a section orthogonal to the longitudinal direction, and a first opening-closing L-shaped part 512 having a plate and plan-view L shape. The internal angle of the L shape is an obtuse angle. The distal end of the first opening-closing L-shaped part 512 is connected with the proximal end of the first opening-closing main part 511, and the proximal end of the first opening-closing L-shaped part 512 is pivotally supported with the distal end of the second support shaft 210b so that these components are relatively rotatable.

The second opening-closing member 530 mainly includes a second opening-closing main part 531 having a semicylindrical shape and a semicircular section at a section orthogonal to the longitudinal direction, and a second opening-closing L-shaped part 532 having a plate and plan-view L shape. Specifically, the second opening-closing member 530 has a shape same as that of the first opening-closing member 510. The distal end of the second opening-closing L-shaped part 532 is connected with the proximal end of the second opening-closing main part 531. The proximal end of the second opening-closing L-shaped part 532 is pivotally supported with the distal end of the first support shaft 210a so that these components are relatively rotatable.

The first opening-closing L-shaped part 512 and the second opening-closing L-shaped part 532 are pivotally supported by a pin 555 at a position between their leading ends and corners so that these components are relatively rotatable. The pin 555 pivotally supports the distal end of the support cover 211 and the proximal end of the holding-movement part 554 together with the first opening-closing L-shaped part 512 and the second opening-closing L-shaped part 532 so that these components are relatively rotatable.

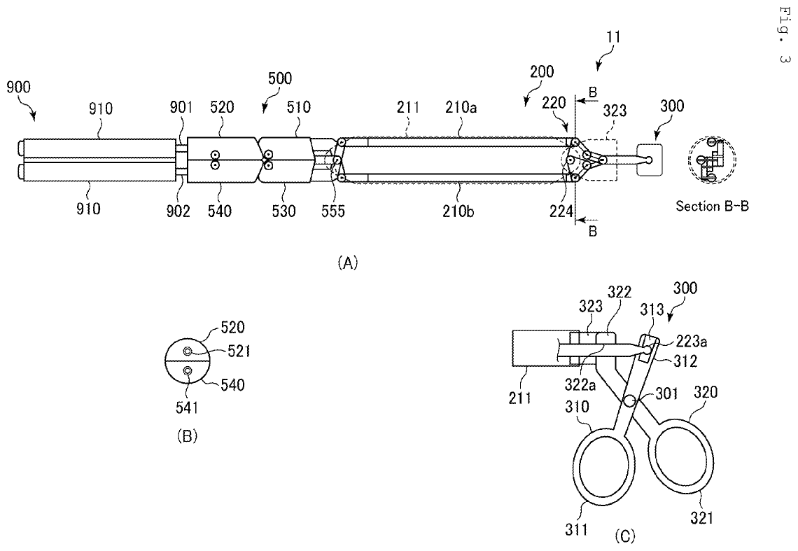

The first holding end part 520 has a semicircular section at a section orthogonal to the longitudinal direction and has a connection hole 521 at an end face on the distal end side (refer to FIG. 3(B)). The proximal end of the first holding end part 520 has an unillustrated protrusion pivotally supported with the distal end of the first opening-closing member 510 so that these components are relatively rotatable. Accordingly, the first holding end part 520 is pivotally supported with the first opening-closing member 510 so that these components are relatively rotatable. The distal end of the first holding operation part 524 is pivotally supported to a surface positioned substantially at the center of the first holding end part 520 in the longitudinal direction and facing the second holding end part 540 so that these components are relatively rotatable.

The second holding end part 540 has a semicircular section at a section orthogonal to the longitudinal direction and has a connection hole 541 at an end face on the distal end side (refer to FIG. 3(B)). The proximal end of the second holding end part 540 has an unillustrated protrusion pivotally supported with the distal end of the second opening-closing member 530 so that these components are relatively rotatable. Accordingly, the second holding end part 540 is pivotally supported with the second opening-closing member 530 so that these components are relatively rotatable. The distal end of the second holding operation part 544 is pivotally supported to a surface positioned substantially at the center of the second holding end part 540 in the longitudinal direction and facing the first holding end part 520 so that these components are relatively rotatable. The proximal end of the first holding operation part 524, the proximal end of the second holding operation part 544, and the distal end of the holding-movement part 554 are pivotally supported so that these components are relatively rotatable.

The sheet cartridge 900 mainly includes a first shaft 901 and a second shaft 902 having cylindrical bar shapes, a sheet 910, and a cover 920.

The first shaft 901 and the second shaft 902 have diameters that allows fixation to prevent the shafts from rotating when inserted into the connection hole 541. The sheet 910 has a rectangular film shape and is made of a material that can be indwelled in the body cavity, one of two long sides of the rectangle serving as a first part 911, the other side serving as a second part 912. The first part 911 is wound around the first shaft 901 but not fixed. Accordingly, the first part 911 is easily removed from the first shaft 901 when the sheet 910 is pulled in a direction departing from the first shaft 901 in a state in which the first part 911 is wound around the first shaft 901. the second part 912 is wound around the second shaft 902 but not fixed. Accordingly, similarly to the first part 911, the second part 912 is easily removed from the second shaft 902.

The cover 920 is a rectangular thin-plate curved in a wound shape and has a proximal end to which a string 921 is attached. The length of the cover 920 in the longitudinal direction is shorter than the first shaft 901 and the second shaft 902 and slightly longer than the sheet 910. The cover 920 is attached over the outer surface of the sheet 910 wound around the first shaft 901 and the second shaft 902. In this state, the cover 920 completely covers the sheet 910 so that the sheet 910 is not contacted from the outside.

The string 921 has a length with which the string 921 extends to the outside of the body cavity through a tube along the first sheet applying device 11 when the sheet cartridge 900 is attached to the first sheet applying device 11.

Sections of the support cover 211 and the first holder 500 orthogonal to the axial direction have substantially same shapes and sizes. Thus, the first sheet applying device 11 has a substantially flush outer peripheral surface from the support cover 211 to the first holder 500 in a state in which the first holder 500 is closed. Accordingly, the first sheet applying device 11 is movable without resistance in a tube 3 to be described later, and body fluid and the like are unlikely to adhere to the outer surface thereof.

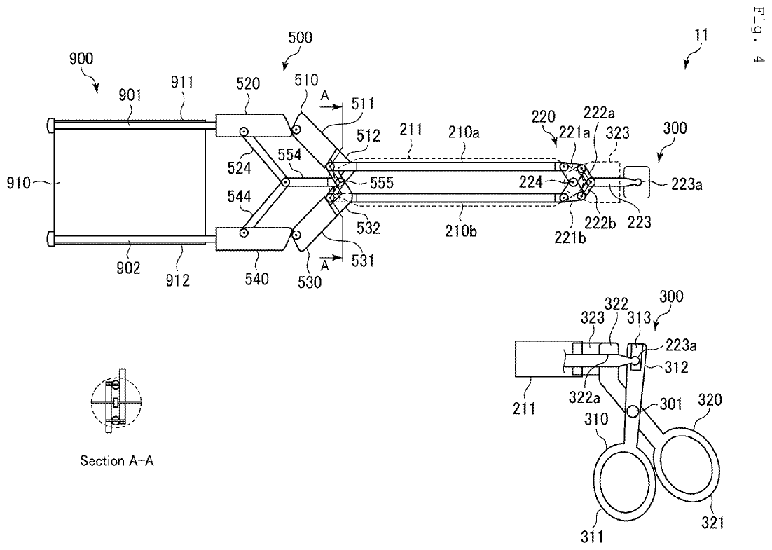

Subsequently, operation of the first sheet applying device 11 will be described below. First, operation to open the sheet 910 will be described below with reference to FIGS. 3 and 4.

When the operator inserts fingers into the finger holes of the first annular part 311 and the second annular part 321 and then grips the first annular part 311 and the second annular part 321, in other words, shortens the interval between the fingers inserted into the finger holes, the first operation lever 310 rotates relative to the second operation lever 320 and the leading end of the first operation bar part 312 approaches the leading end of the second operation bar part 322. Then, the ball-shaped proximal end 223a moves in the bottomed cylinder hole 313, and the swing movement part 223 is moved by pressing toward the distal end side. Accordingly, the swing movement part 223 presses the first swing-support part 221a and the second swing-support part 221b toward the distal end side through the first swing operation part 222a and the second swing operation part 222b. As described above, the proximal end of the operation cover 323 is fixed to the leading end of the second operation bar part 322, and the distal end thereof pivotally supports the first swing-support part 221a and the second swing-support part 221b. Thus, as the swing movement part 223 presses the first swing operation part 222a and the second swing operation part 222b toward the distal end side, the first swing-support part 221a and the second swing-support part 221b rotate about the pin 224.

Then, the corners of the first swing-support part 221a and the second swing-support part 221b positioned on the distal end side press the first support shaft 210a and the second support shaft 210b toward the distal end side. Accordingly, the first support shaft 210a and the second support shaft 210b move in the longitudinal direction of the first supporter. As described above, the proximal end side of the support cover 211 is pivotally supported together with the distal ends of the first swing-support part 221a and the second swing-support part 221b by the pin 224 so that these components are relatively rotatable, and the distal end side of the support cover 211 is pivotally supported together with the first opening-closing L-shaped part 512 and the second opening-closing L-shaped part 532 by the pin 555 so that these components are relatively rotatable. Thus, as the first support shaft 210a being pressed toward the distal end side presses the proximal end of the second opening-closing L-shaped part 532, the second opening-closing member 530 rotates about the pin 555 and becomes separated from the first opening-closing member 510, and in addition, as the second support shaft 210b being pressed toward the distal end side presses the proximal end of the first opening-closing L-shaped part 512, the first opening-closing member 510 rotates about the pin 555 and becomes separated from the second opening-closing member 530. Accordingly, the first holding end part 520 and the second holding end part 540 are separated from each other. As described above, the first holding end part 520 and the second holding end part 540 are connected with the pin 555 through the first holding operation part 524, the second holding operation part 544, and the holding-movement part 554. Thus, the first holding end part 520 and the second holding end part 540 are separated in parallel to each other. Accordingly, the first shaft 901 and the second shaft 902 being rotatably held at the first holding end part 520 and the second holding end part 540, respectively, are separated in parallel to each other. Accordingly, the sheet 910 being wound around the first shaft 901 and the second shaft 902 is expanded or unfolded.

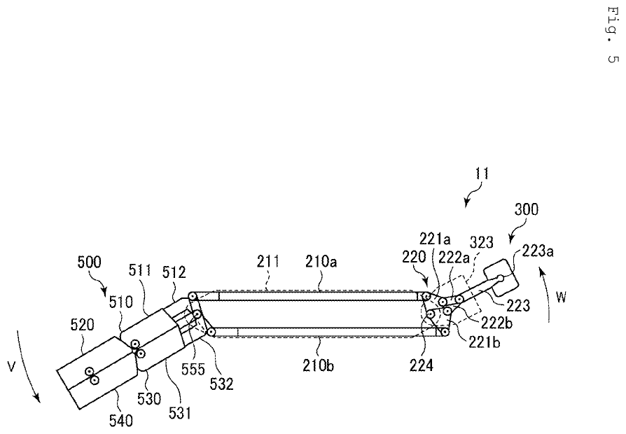

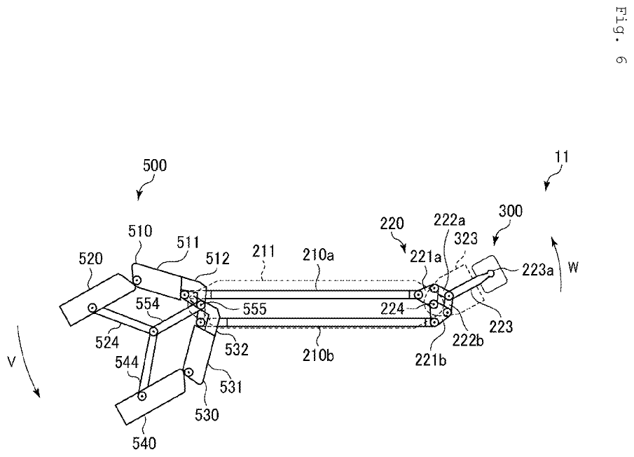

Subsequently, operation to swing or oscillate the first holder 500 will be described below with reference to FIGS. 5 and 6. First, the operator grasps the handle 300 with one hand and then grasps the support cover 211 with the other hand. Then, as the handle 300 is swung about the pin 224 relative to the support cover 211 in a direction W as illustrated in FIG. 5 in a plane including the first support shaft 210a and the second support shaft 210b, the swing movement part 223 presses the first swing-support part 221a toward the distal end side through the first swing operation part 222a and pulls the second swing-support part 221b toward the proximal end side through the second swing operation part 222b. As described above, the proximal end side of the support cover 211 is pivotally supported together with the distal ends of the first swing-support part 221a and the second swing-support part 221b by the pin 224 so that these components are relatively rotatable, and the distal end side of the support cover 211 is pivotally supported together with the first opening-closing L-shaped part 512 and the second opening-closing L-shaped part 532 by the pin 555 so that these components are relatively rotatable. Thus, the first support shaft 210a being pressed toward the distal end side presses the proximal end of the second opening-closing L-shaped part 532, and the second support shaft 210b being pulled toward the proximal end side pulls the proximal end of the first opening-closing L-shaped part 512. Accordingly, the first holder 500 rotates about the pin 555 in a direction V as illustrated in FIG. 5. Specifically, the first opening-closing member 510 and the second opening-closing member 530 swing in the plane including the first support shaft 210a and the second support shaft 210b.

Note that although the configuration in which the first opening-closing member 510 and the second opening-closing member 530 swing while being closed is described above with reference to FIG. 5, the first opening-closing member 510 and the second opening-closing member 530 can swing while being opened as illustrated in FIG. 6 through the same process.

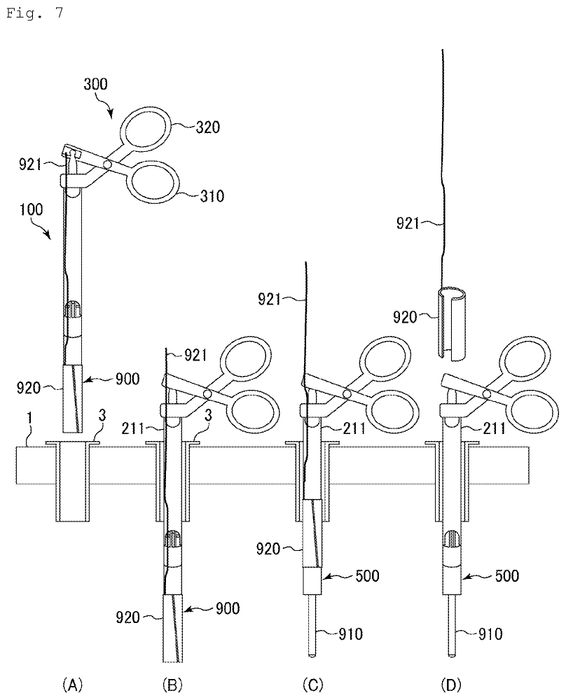

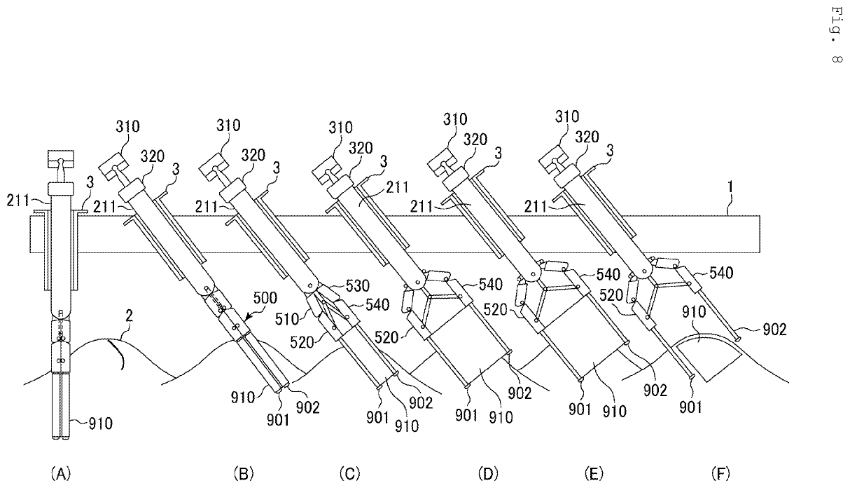

Subsequently, a sheet applying method of applying the sheet 910 to an affected part in the body cavity by the first sheet applying system 10 will be described below with reference to FIGS. 7 and 8.

As illustrated in FIG. 7, the tube 3 is provided through a body wall 1 (refer to FIG. 7(A)). The first sheet applying device 11 to which the sheet cartridge 900 is attached is inserted into the tube 3 so that the sheet cartridge 900 protrudes into the body cavity from a leading end of the tube 3 (refer to FIG. 7(B)). In this state, the string 921 extends to the outside of the body cavity through the tube 3 along the first sheet applying device 11.

Subsequently, the operator pulls the string 921 extending to the outside of the body cavity. Accordingly, the cover 920 is displaced from the sheet cartridge 900 and moved toward the proximal end side. In this case, the cover 920, which is curved in a wound shape, is movable toward the proximal end side while being wound around the outer periphery of the support cover 211 (refer to FIG. 7(C)). Then, the cover 920 is taken out of the body (refer to FIG. 7(D)).

FIG. 8(A) illustrates the sheet cartridge 900 and the first sheet applying device 11 from which the cover 920 is removed. In this state, the sheet 910 protrudes into the body cavity from the leading end of the tube 3. In this state, the operator operates the handle 300 to swing or oscillate the first holder 500 so that the sheet cartridge 900 contacts or moves close to a target 2 (refer to FIG. 8(B)). Typically, the inside of the body cavity is extremely narrow, and thus it is difficult to freely move the first sheet applying device 11 in some cases. However, the first sheet applying device 11 can swing or oscillate the first holder 500. Thus, even in a little space, the first holder 500 can be swung or oscillated to easily move the sheet 910 to the target 2.

When the operator grips the first annular part 311 and the second annular part 321, the first holding end part 520 and the second holding end part 540 become separated from each other and the sheet 910 is expanded (refer to FIG. 8(C)). Then, after the sheet 910 is expanded to some extent, part of the sheet 910 is made contact with the target (refer to FIG. 8(D)). In this case, a substantially central part of the sheet 910 is preferably made contact with the target. Note that the substantially central part includes a central part. The sheet 910 has a moisture absorption property and thus is unlikely to be displaced from a target containing water. Since the sheet 910 expands in a rectangular shape as the first shaft 901 and the second shaft 902 are separated in parallel to each other, part of the sheet 910 can be easily and reliably made contact with the target by the operator.

Subsequently, the operator further expands the sheet 910. As described above, the first part 911 and the second part 912 are wound around the first shaft 901 and the second shaft 902 but not fixed. Thus, as the operator further grips the first annular part 311 and the second annular part 321, the first part 911 and the second part 912 are easily and automatically removed from the first shaft 901 and the second shaft 902, and accordingly, the sheet 910 comes off the first shaft 901 and the second shaft 902 (refer to FIG. 8(E)). In this case, since part of the sheet 910 is in contact with the target as described above (refer to FIG. 8(D)), the sheet 910 is accurately applied to the target without being displaced from the target. In other words, the sheet 910 can be applied to the target while being expanded. Accordingly, the sheet 910 can be easily applied to the target (refer to FIG. 8(F)).

According to the present embodiment, the sheet 910 can be easily applied to the target.

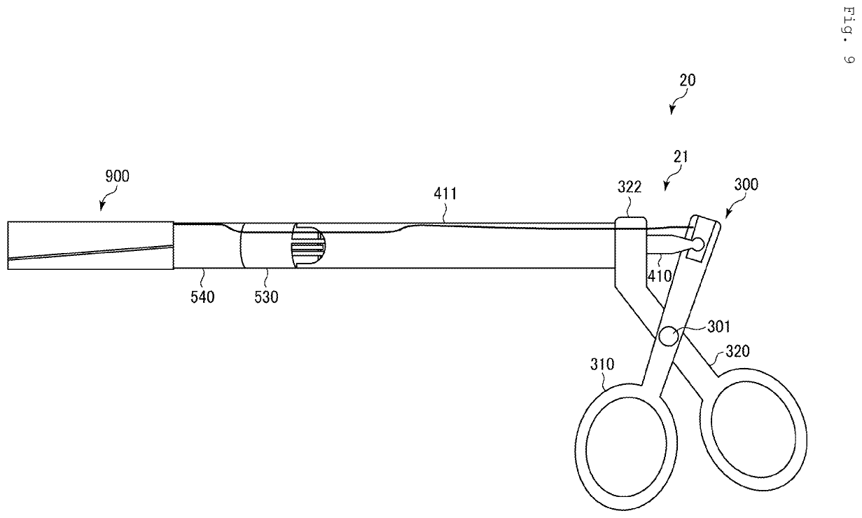

A second sheet applying system 20 according to a second embodiment will be described below with reference to FIGS. 9 to 11. Any component same as that in the first embodiment is denoted by the same reference sign, and description thereof is omitted.

The second sheet applying system 20 mainly includes a second sheet applying device 21 and the sheet cartridge 900. The second sheet applying device 21 mainly includes a second supporter 400 and the first holder 500. In the second sheet applying system 20, the configuration of the second supporter 400 is mainly different from that in the first embodiment. Thus, the second supporter 400 will be mainly described below.

The second supporter 400 mainly includes a support shaft 410, a third swing-support part 411a, a fourth swing-support part 411b, a support cover 411, and the handle 300. The support shaft 410 has a straight cylindrical bar shape extends and is movable from the proximal end side of the second sheet applying system 20 toward the distal end side thereof, in other words, in the longitudinal direction of the second supporter 400. A proximal end 423a of the support shaft 410 has a ball shape and is connected with the handle 300. This connection means is same as that in the first embodiment, and thus description thereof is omitted. The third swing-support part 411a and the fourth swing-support part 411b are thin plates having the same length in the longitudinal direction. The proximal ends of the third swing-support part 411a and the fourth swing-support part 411b are pivotally supported to the distal end of the support shaft 410 so that these components are relatively rotatable. The distal end of the third swing-support part 411a is pivotally supported with the proximal end of the second opening-closing L-shaped part 532 so that these components are relatively rotatable, and the distal end of the fourth swing-support part 411b is pivotally supported with the proximal end of the first opening-closing L-shaped part 512 so that these components are relatively rotatable. The support cover 411 has no semicircular cutout at the proximal end and is fixed to the leading end of the second operation bar part 322, which is difference from the first embodiment. The other configuration is same as that in the first embodiment, and thus description thereof is omitted.

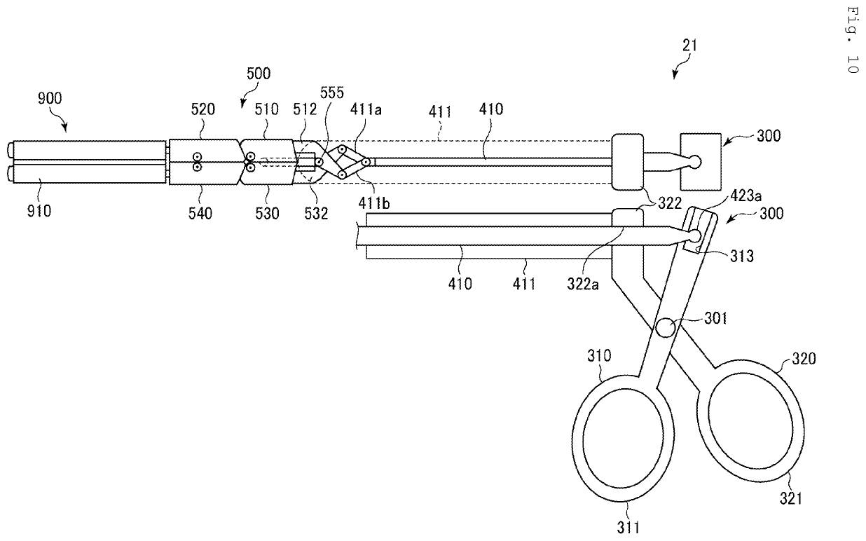

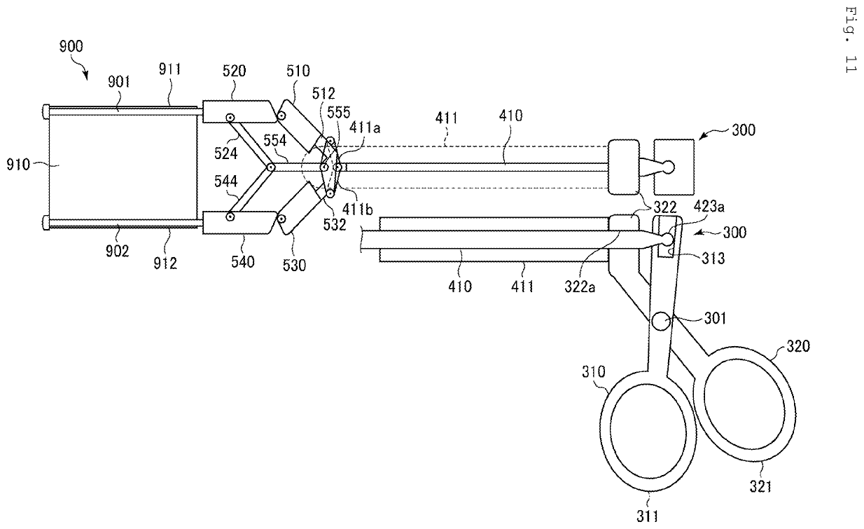

Subsequently, operation of the second sheet applying device 21 will be described below with reference to FIGS. 10 and 11.

When the operator grips the first annular part 311 and the second annular part 321, the ball-shaped proximal end 423a moves in the bottomed cylinder hole 313, and the support shaft 410 is moved by pressing toward the distal end side. Accordingly, the support shaft 410 presses the proximal end of the second opening-closing L-shaped part 532 and the proximal end of the first opening-closing L-shaped part 512 toward the distal end side through the third swing-support part 411a and the fourth swing-support part 411b. Accordingly, the first opening-closing member 510 and the second opening-closing member 530 rotate about the pin 555 to become separated from each other. Then, the first shaft 901 and the second shaft 902 are separated in parallel to each other, and the sheet 910 is expanded or unfolded.

According to the present embodiment, with a simple configuration, the sheet 910 can be applied to a target, and effects same as those of the first embodiment are achieved.

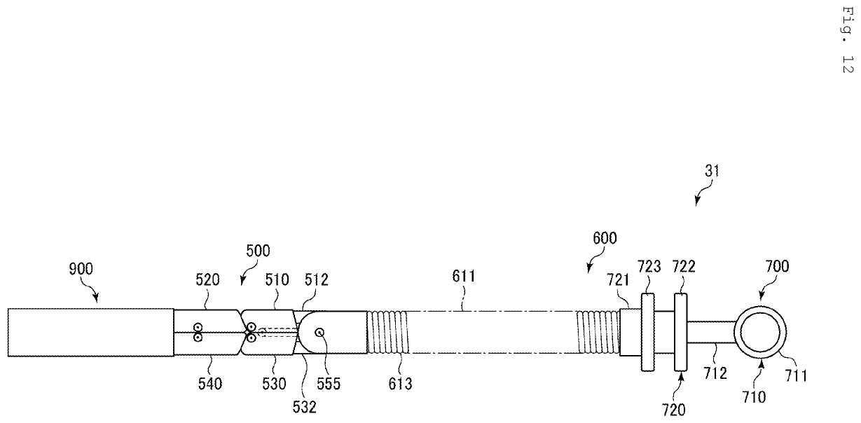

A third sheet applying system 30 according to a third embodiment will be described below with reference to FIGS. 12 to 15. Any component same as that in the first and second embodiments is denoted by the same reference sign, and description thereof is omitted.

The third sheet applying system 30 mainly includes a third sheet applying device 31 and the sheet cartridge 900. The third sheet applying device 31 mainly includes a third supporter 600 and the first holder 500. In the third sheet applying system 30, the configuration of the third supporter 600 is mainly different from that in the first embodiment. Thus, the third supporter 600 will be mainly described below.

The third supporter 600 mainly includes a support wire 610, a fifth swing-support part 611a, a sixth swing-support part 611b, a support cover 611, and a handle 700. The support wire 610 is flexible, has a cylindrical bar shape, and extends and is movable from the proximal end side of the third sheet applying system 30 toward the distal end side thereof, in other words, in the longitudinal direction of the third supporter 600. The proximal end of the support wire 610 is connected with the handle 700. The fifth swing-support part 611a and the sixth swing-support part 611b are thin plates having the same length in the longitudinal direction. The proximal ends of the fifth swing-support part 611a and the sixth swing-support part 611b are pivotally supported to the distal end of the support wire 610 so that these components are relatively rotatable. The distal end of the fifth swing-support part 611a is pivotally supported with the proximal end of the second opening-closing L-shaped part 532 so that these components are relatively rotatable, and the distal end of the sixth swing-support part 611b is pivotally supported with the proximal end of the first opening-closing L-shaped part 512 so that these components are relatively rotatable. The support cover 611 mainly includes a cover bending part 613 and a cover distal end part 614. The cover bending part 613 is a flexible tube made of a helically wound wire. The cover distal end part 614 has a configuration same as that of the distal end of the support cover 211 according to the first embodiment, and the proximal end thereof is connected with the distal end of the cover bending part.

The handle 700 mainly includes an operation part 710 and a grasping part 720. The operation part 710 mainly includes a ring 711 and a wire fixation part 712 protruding straight from the outer periphery of the ring. The grasping part 720 mainly includes a main grasping part 721 having a cylindrical shape, a proximal end side flange 722 protruding at right angle from the outer periphery of the proximal end of the main grasping part 721, and a center flange 723 protruding at right angle from the outer periphery of the main grasping part 721 substantially at the center in the longitudinal direction. The support wire 610 penetrates to the inner periphery of the grasping part 720 and is fixed to the main grasping part 721.

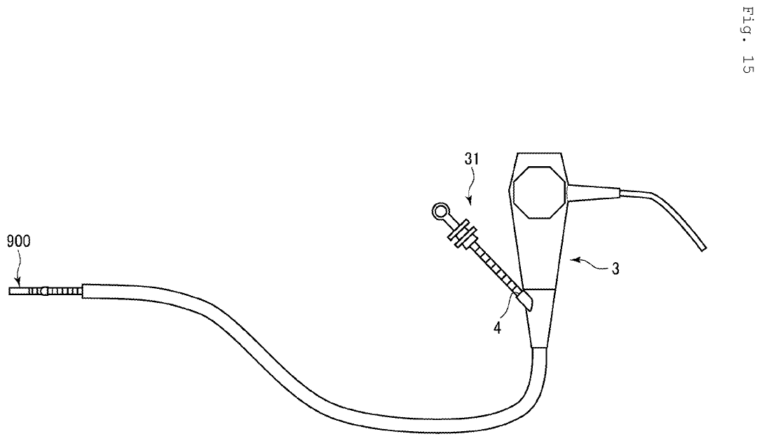

With the above-described configuration, the third supporter 600 can be bent. Accordingly, the third sheet applying device 31 can be used together with an endoscope device 3 that is flexible (refer to FIG. 15). Specifically, the third sheet applying device 31 can be inserted through a forceps opening 4 of the endoscope device 3, and the sheet 910 can be applied to a target while the positional relation between the target and the sheet 910 is observed through the endoscope device 3.

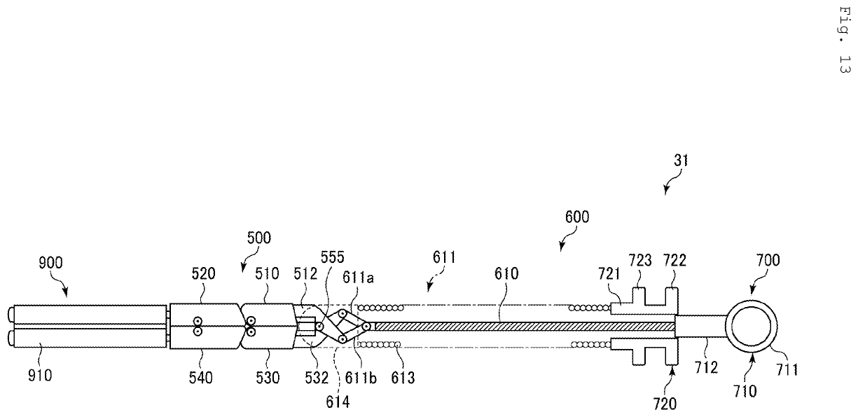

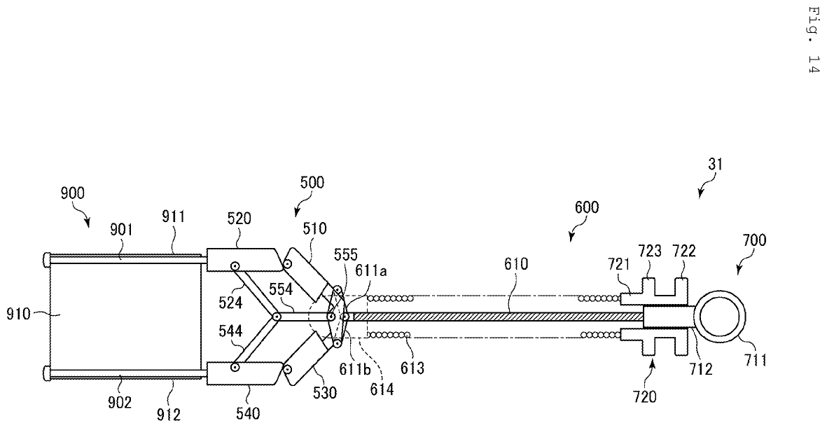

Subsequently, operation of the third sheet applying device 31 will be described below with reference to FIGS. 13 and 14.

As the operator presses the operation part 710 toward the distal end, the support wire 610 is moved by pressing toward the distal end side. Accordingly, the support wire 610 presses the proximal end of the second opening-closing L-shaped part 532 and the proximal end of the first opening-closing L-shaped part 512 toward the distal end side through the fifth swing-support part 611a and the sixth swing-support part 611b. Consequently, the first opening-closing member 510 and the second opening-closing member 530 rotate about the pin 555 to become separated from each other. Then, the first shaft 901 and the second shaft 902 are separated in parallel to each other, and the sheet 910 is expanded or unfolded.

According to the present embodiment, effects same as those of the first and second embodiments are achieved. In addition, the third supporter 600 is bendable and thus can be used together with the flexible endoscope device 3. Specifically, the sheet 910 can be applied to a target while the positional relation between the target and the sheet 910 is observed through the endoscope device 3.

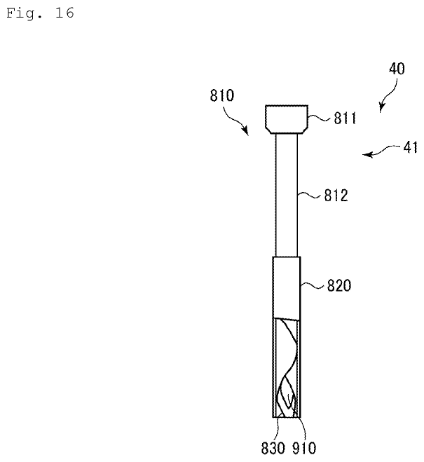

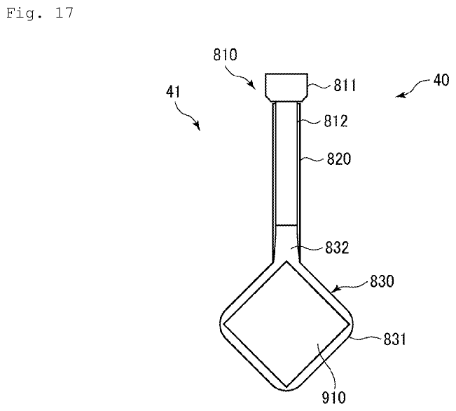

A fourth sheet applying system 40 according to a fourth embodiment will be described below with reference to FIGS. 16 to 18. Any component same as that in the first to third embodiments is denoted by the same reference sign, and description thereof is omitted.

The fourth sheet applying system 40 mainly includes a fourth sheet applying device 41. The fourth sheet applying device 41 mainly includes a fourth supporter 810, a protector 820, and a holder 830.

The fourth supporter 810 mainly includes a knob 811 and a bar part 812. The knob 811 has a cylindrical shape, and the distal end side thereof is connected with the bar part 812. The bar part 812 has a cylindrical shape, and the distal end side thereof is connected with the holder 830.

The holder 830 is made of a thin-plate film that can be wound, and includes a holding part 831 having a substantially square shape, and a tongue part 832 protruding outward on a diagonal line from a corner of the holding part 831. The holding part 831 is slightly larger than the sheet 910. The tongue part 832 is connected with the distal end of the bar part 812.

The protector 820 is shaped in a cylinder having an inner diameter larger than the bar part 812 and smaller than the knob 811 and can store, on the inner periphery thereof, the holder 830 being wound and the bar part 812.

The holding part 831 holds the sheet 910 being wound and enclosed on the inner periphery side while the sheet 910 is placed on one surface of the holding part 831. Then, the holding part 831 in which the sheet 910 is enclosed is stored on the inner periphery of the protector 820 (refer to FIG. 16). This completes the fourth sheet applying device 41. Note that the holding part 831 has elasticity, and thus is pressed in an opening direction while being wound. With this pressing force, the holding part 831 is held on the inner periphery of the protector 820, and the holding part 831 automatically expands when removed from the protector 820. Then, since the holding part 831 is not firmly fixed to the sheet 910, the sheet 910 automatically and easily falls from the holding part 831 when the holding part 831 opens.

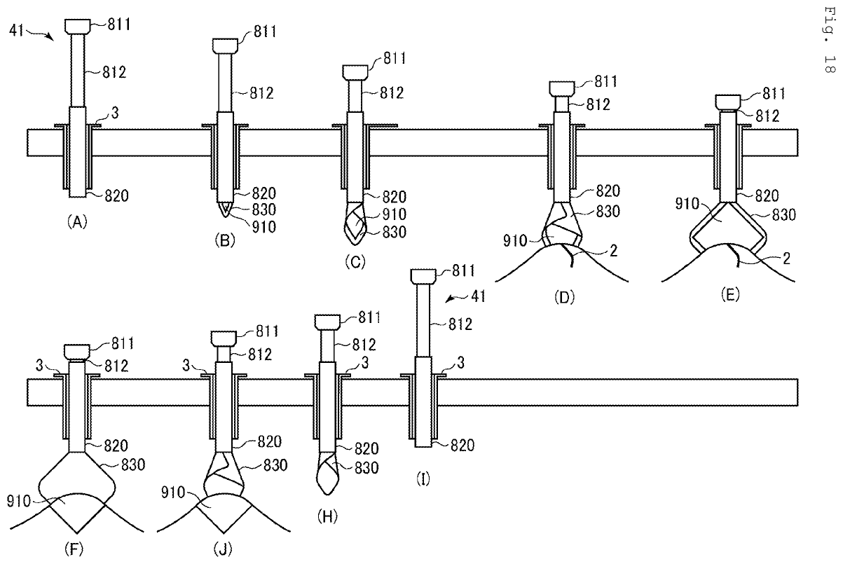

Subsequently, a sheet applying method of applying the sheet 910 to an affected part in the body cavity by the fourth sheet applying system 40 will be described below with reference to FIG. 18.

As illustrated in FIG. 18(A), the tube 3 is provided through the body wall 1. The fourth sheet applying device 41 is inserted into the tube 3. Subsequently, the knob 811 is moved by pressing toward the distal end side so that part of the sheet 910 protrudes into the body cavity from a leading end of the protector 820 (refer to FIG. 18(B)). Then, as the knob 811 is further moved by pressing, the holding part 831 protruding from the leading end of the protector 820 starts expanding (refer to FIG. 18(C)). As the holding part 831 expands, the sheet 910 expands. After the holding part 831 is expanded to some extent, the operator operates the fourth sheet applying device 41 to move the sheet 910 to above the target 2 (refer to FIG. 18(D)). Then, as the knob 811 is further moved by pressing, the holding part 831 completely protrudes from the leading end of the protector 820 and expands, and accordingly, the sheet 910 expands (refer to FIG. 18(E)). The holding part 831 is not firmly fixed to the sheet 910 as described above, and thus when the holding part 831 opens to some extent, the sheet 910 is automatically and easily ejected or falls from the holding part 831, and adheres to the target (refer to FIG. 18(F)). Accordingly, the sheet 910 can be easily applied to the target.

Subsequently, as the knob 811 is pulled toward the proximal end side, the holding part 831 enters the inner periphery of the protector 820 (refer to FIG. 18(G)). Specifically, a connection site of the holding part 831 and the tongue part 832 is gently formed or the angle between two sides of the holding part 831 sandwiching the tongue part 832 is appropriately determined, and thus the holding part 831 enters the inner periphery of the protector 820 while being easily wound (refer to FIG. 18(H)). Then, after the holding part 831 is completely stored on the inner periphery of the protector 820, the fourth sheet applying device 41 is pulled out of the tube 3 (refer to FIG. 18(I)). This completes treatment.

According to the present embodiment, effects same as those of the first to third embodiments are achieved. In addition, the fourth sheet applying device 41 has a simple configuration and thus can be inexpensively manufactured in a short period.

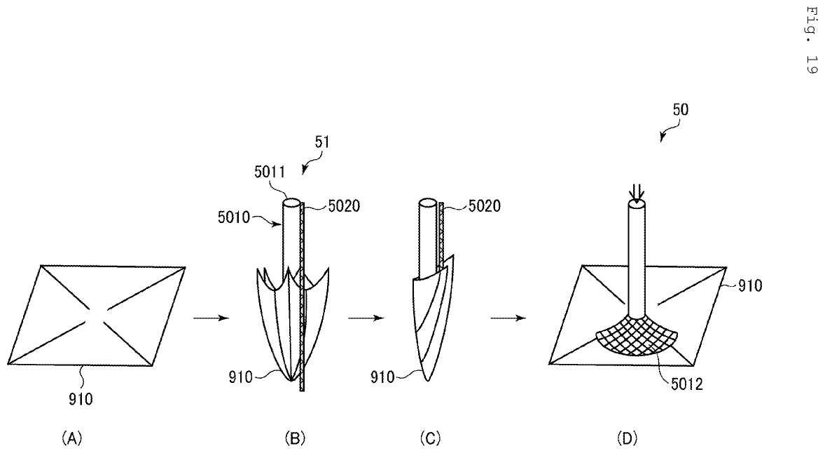

A fifth sheet applying system 50 according to a fifth embodiment will be described below with reference to FIG. 19. Any component same as that in the first to fourth embodiments is denoted by the same reference sign, and description thereof is omitted.

The fifth sheet applying system 50 mainly includes a fifth sheet applying device 51. The fifth sheet applying device 51 mainly includes a fifth main supporter 5010 and a sub supporter 5020. The fifth main supporter 5010 mainly includes a cylinder 5011 and a pusher 5012. The pusher 5012 is a balloon 5012 stored inside the cylinder 5011 from the distal end side. The balloon 5012 is pressed to swell, and expands, in other words, swells when protruding from the distal end of the cylinder 5011. For example, the handle 300, which is not illustrated, is attached to the proximal end of the cylinder 5011. As the handle 300 is operated, the balloon 5012 protrudes from the distal end of the cylinder 5011 and swells. The sub supporter 5020 is a bar.

Subsequently, a means for attaching the sheet 910 to the fifth sheet applying device 51 will be described below. In FIG. 19(A), the fifth main supporter 5010 is placed at the center of the sheet 910 being expanded. In FIG. 19(B), the sheet 910 is folded in an umbrella shape, the sub supporter 5020 is placed thereon, and the sheet 910 is sandwiched and fixed between the fifth main supporter 5010 and the sub supporter 5020. Accordingly, the fifth main supporter 5010 holds the sheet 910. In other words, the fifth main supporter 5010 serves as a holder in the present embodiment. In FIG. 19(C), the sheet 910 is wound to enclose the fifth main supporter 5010 and the sub supporter 5020. Then, the cover 920, which is not illustrated, is attached thereon. The sheet 910 is not firmly fixed to the fifth main supporter 5010 and the sub supporter 5020 but only wound. In this manner, the sheet 910 is attached to the fifth sheet applying device 51.

Subsequently, a sheet applying method of applying the sheet 910 to an affected part in the body cavity by the fifth sheet applying system 50 will be described below with reference to FIG. 19(D). The fifth sheet applying device 51 is inserted into the tube 3, which is not illustrated, so that the sheet 910 entirely protrudes into the body cavity from the leading end of the tube 3. Then, the cover 920 is removed by the above-described means. Subsequently, the sub supporter 5020 is pulled out of the body, and then the handle 300 is operated so that the balloon 5012 protrudes from the distal end of the cylinder 5011 and swells. Accordingly, the sheet 910 being wound around the fifth main supporter 5010 expands (refer to FIG. 19(D)). The sheet 910 is not firmly fixed to the fifth main supporter 5010 and the sub supporter 5020 as described above, and thus when the balloon 5012 opens to some extent, the sheet 910 is automatically and easily ejected or falls from the fifth sheet applying device 51, and adheres to the target (refer to FIG. 19(D)). Accordingly, the sheet 910 can be easily applied to the target. Thereafter, the handle 300 is operated to store the balloon 5012 inside the cylinder 5011. Then, after the balloon 5012 is completely stored on the inner periphery of the cylinder 5011, the fifth sheet applying device 51 is pulled out of the tube 3. This completes treatment.

According to the present embodiment, effects same as those of the first to third embodiments are achieved. In addition, the fifth sheet applying device 51 has a simple configuration and thus can be inexpensively manufactured in a short period.

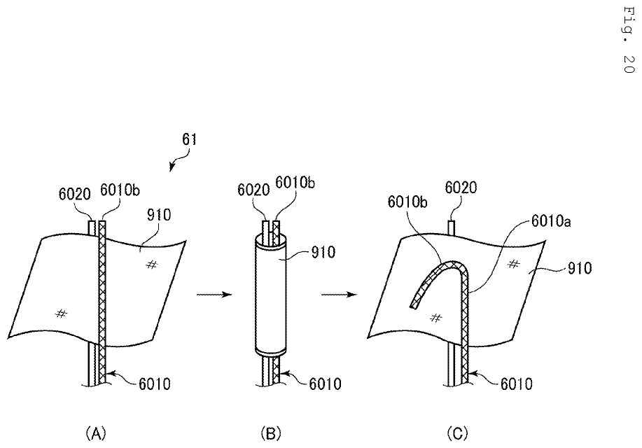

A sixth sheet applying system 60 according to a sixth embodiment will be described below with reference to FIG. 20. Any component same as that in the first to fifth embodiments is denoted by the same reference sign, and description thereof is omitted.

The sixth sheet applying system 60 mainly includes a sixth sheet applying device 61. The sixth sheet applying device 61 mainly includes a sixth main supporter 6010 and a sub supporter 6020. The sixth main supporter 6010 mainly includes a bar part 6010a and a bending part 6010b. The bar part 6010a is a member bendable in accordance with bending of the endoscope when inserted into a forceps opening of a soft endoscope. The bending part 6010b is a member having a section substantially same as that of the bar part, attached to the distal end of the bar part 6010a, and bendable by, for example, the handle 300, which is not illustrated. A rotation knob, which is not illustrated, is provided at the proximal end of the bar part 6010a. A wire extends through inside of the bar part 6010a from the rotation knob to the bending part 6010b. When the rotation knob is rotated clockwise or anticlockwise, the bending part 6010b is bent in the range of 270° with respect to a straight state. The sub supporter 6020 is a bar.

Subsequently, a means for attaching the sheet 910 to the sixth sheet applying device 61 will be described below. In FIG. 20(A), the sheet 910 is sandwiched between the sixth main supporter 6010 and the sub supporter 6020. In FIG. 20(B), the sheet 910 is wound to enclose the sixth main supporter 6010 and the sub supporter 6020. Then, the cover 920, which is not illustrated, is attached thereon. Accordingly, the sixth main supporter 6010 holds the sheet 910. In other words, the sixth main supporter 6010 serves as a holder in the present embodiment. Note that the sheet 910 is not firmly fixed to the sixth main supporter 6010 and the sub supporter 6020 but only wound. Through the above-described process, the sheet 910 is attached to the sixth sheet applying device 61.

Subsequently, a sheet applying method of applying the sheet 910 to an affected part in the body cavity by the sixth sheet applying system 60 will be described below with reference to FIG. 20(C). The sixth sheet applying device 61 is inserted into the tube 3, which is not illustrated, so that the sheet 910 entirely protrudes into the body cavity from the leading end of the tube 3. Then, the cover 920 is removed by the above-described means. Subsequently, the rotation knob is operated to bend the bending part 6010b. Accordingly, the sheet 910 wound around the sixth main supporter 6010 and the sub supporter 6020 expands (refer to FIG. 20(C)). The sheet 910 is not firmly fixed to the sixth main supporter 6010 and the sub supporter 6020 as described above, and thus when the bending part 6010b is bent to some extent, the sheet 910 is automatically and easily ejected or falls from the sixth main supporter 6010 and the sub supporter 6020, and adheres to the target. Accordingly, the sheet 910 can be easily applied to the target. Thereafter, the rotation knob is operated to return the bending part 6010b to the straight state. Then, the sixth sheet applying device 61 is pulled out of the tube 3. This completes treatment.

According to the present embodiment, effects same as those of the first to fourth embodiments are achieved. In addition, the sixth sheet applying device 61 has a simple configuration and thus can be inexpensively manufactured in a short period.

Note that the sheet 910 according to the present embodiment may be same as that of another embodiment, but is preferably more self-expandable than that of the other embodiment.

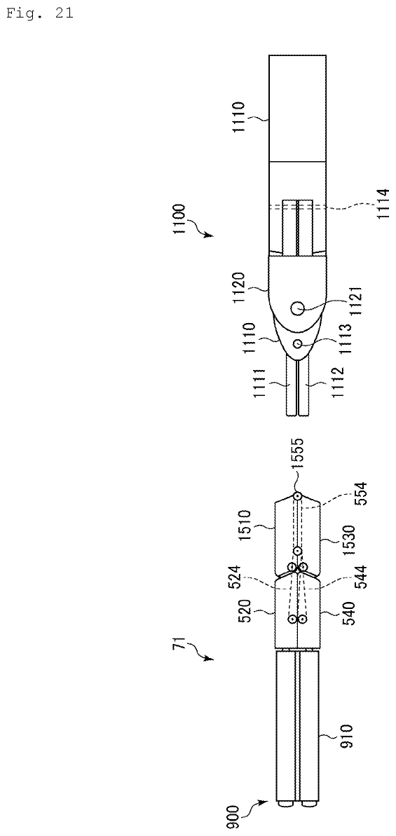

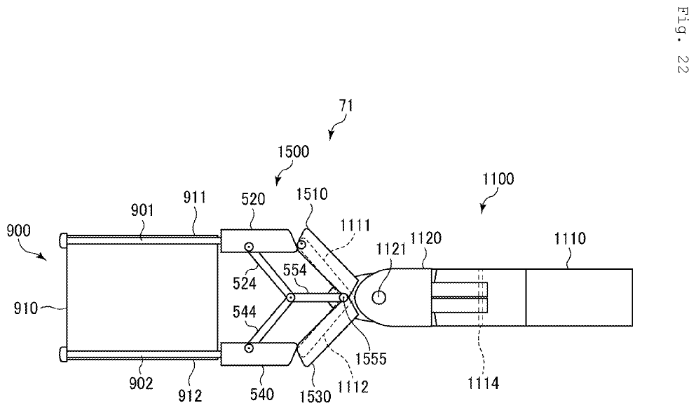

A seventh sheet applying system 70 according to a seventh embodiment will be described below with reference to FIGS. 21 and 22. Any component same as that in the first to sixth embodiments is denoted by the same reference sign, and description thereof is omitted.

The seventh sheet applying system 70 mainly includes a seventh sheet applying device 71 that is attachable to a surgical operation support robot 1100.

The surgical operation support robot 1100 mainly includes a robot proximal end part 1110, a robot middle part 1120, and a robot distal end part 1110. The robot middle part 1120 is pivotally supported to the robot proximal end part 1110 so that these components are rotatable through a pin 1114, and the robot distal end part 1110 is pivotally supported to the robot middle part 1120 so that these components are rotatable through a pin 1121. Jaws 1111 and 1112 are provided at the distal end of the robot distal end part 1110. The jaws 1111 and 1112 are pivotally supported to the robot distal end part 1110 so that these components are rotatable through a pin 1113. The robot middle part 1120, the robot distal end part 1110, and the jaws 1111 and 1112 are rotated by the operator through an unillustrated console included in the surgical operation support robot 1100.

The seventh sheet applying device 71 mainly includes a second holder 1500. The second holder 1500 mainly includes a twenty-first opening-closing member 1510, a twenty-second opening-closing member 1530, the first holding end part 520, the second holding end part 540, and the first holding operation part 524, the second holding operation part 544, and the holding-movement part 554 having thin-plate bar shapes. The configurations of the first holding end part 520, the second holding end part 540, the first holding operation part 524, the second holding operation part 544, and the holding-movement part 554 are same as those in the first embodiment, and thus description thereof is omitted.

The twenty-first opening-closing member 1510 and the twenty-second opening-closing member 1530 are each a bar having a semicircular section at a section orthogonal to the longitudinal direction and having a hole (connection part) extending in the longitudinal direction of the member. The jaws 1111 and 1112 are inserted into these holes and fixed at positions where the pin 1113 has an axis identical to that of a pin 1555. The twenty-first opening-closing member 1510 and the twenty-second opening-closing member 1530 are pivotally supported by the pin 1555 at the proximal ends so that these components are relatively rotatable. Connection between each of the twenty-first opening-closing member 1510 and the twenty-second opening-closing member 1530 and the corresponding one of the first holding end part 520 and the second holding end part 540 is same as that in the first embodiment. Operation to swing or oscillate the second holder 1500 is achieved as the robot middle part 1120 rotates relative to the robot proximal end part 1110 through the pin 1114 or as the robot distal end part 1110 rotates relative to the robot middle part 1120 through the pin 1121.

Subsequently, operation of the seventh sheet applying device 71 will be described below. When the operator operates the unillustrated console to rotate and open the jaws 1111 and 1112, the twenty-first opening-closing member 1510 rotates about the pin 1555 and becomes separated from the twenty-second opening-closing member 1530. Accordingly, the first holding end part 520 and the second holding end part 540 are separated from each other. Similarly to the first embodiment, when the first holding end part 520 and the second holding end part 540 are separated in parallel to each other, the first shaft 901 and the second shaft 902 are separated in parallel to each other and the sheet 910 being wound around the first shaft 901 and the second shaft 902 is expanded or unfolded. A sheet applying method of applying the sheet 910 to an affected part in the body cavity by the seventh sheet applying system 70 is same as that of the first embodiment except that the twenty-first opening-closing member 1510 and the twenty-second opening-closing member 1530 are opened and closed through the jaws 1111 and 1112, and thus description thereof is omitted.

According to the present embodiment, effects same as those of the first to fourth embodiments are achieved. In addition, the sheet 910 can be easily applied to the target by using the surgical operation support robot 1100.

Note that, in the present embodiment, a connection part through which the jaws 1111 and 1112 are connected is not limited to a hole but may be any member through which the twenty-first opening-closing member 1510 and the twenty-second opening-closing member 1530 can be connected with the jaws 1111 and 1112.

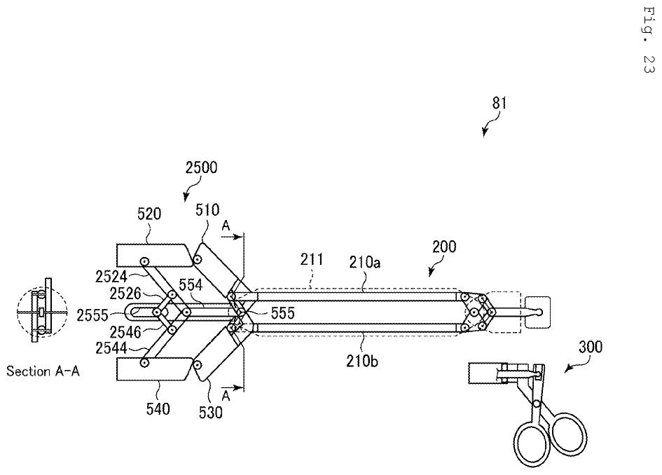

Subsequently, an eighth sheet applying system 80 according to an eighth embodiment of the present invention will be described below with reference to FIG. 23. Any component same as that in the first to seventh embodiments is denoted by the same reference sign, and description thereof is omitted. The eighth sheet applying system 80 mainly includes an eighth sheet applying device 81 and the sheet cartridge 900.

The eighth sheet applying device 81 mainly includes the first supporter 200 and an eighty-first holder 2500. The first supporter 200 has a configuration same as that in the first embodiment and mainly includes the first support shaft 210a, the second support shaft 210b, the first swing mechanism 220, the support cover 211, and the handle 300. The handle 300 has a configuration same as that in the first embodiment, and thus description thereof is omitted.

The eighty-first holder 2500 mainly includes the first opening-closing member 510, the second opening-closing member 530, the first holding end part 520, the second holding end part 540, and an eighty-first holding operation part 2524, an eighty-second holding operation part 2544, an eighty-first holding support part 2526, an eighty-second holding support part 2546, and a holding-movement part 2554 having thin-plate bar shapes. The configurations of the first opening-closing member 510, the second opening-closing member 530, the first holding end part 520, and the second holding end part 540 are same as those in the first embodiment, and thus description thereof is omitted.

The holding-movement part 2554 has a hole through which the pin 555 can penetrate on the proximal end side, and has a long hole 2555 on the distal end side. The pin 555 pivotally supports the distal end of the support cover 211 and the proximal end of the holding-movement part 2554 together with the first opening-closing L-shaped part 512 and the second opening-closing L-shaped part 532 so that these components are relatively rotatable.

The distal end of the eighty-first holding operation part 2524 is pivotally supported to a surface positioned slightly on the distal end side of the center of the first holding end part 520 in the longitudinal direction and facing the second holding end part 540 so that these components are relatively rotatable. The distal end of the eighty-second holding operation part 2544 is pivotally supported to a surface positioned slightly on the distal end side of the center of the second holding end part 540 in the longitudinal direction and facing the first holding end part 520 so that these components are relatively rotatable. The proximal end of the eighty-first holding operation part 2524 and the proximal end of the eighty-second holding operation part 2544 are pivotally supported to substantially the center of the holding-movement part 2554 in the longitudinal direction so that these components are relatively rotatable. The proximal end of the eighty-first holding support part 2526 is pivotally supported on the proximal end side of the center of the eighty-first holding operation part 2524 so that these components are relatively rotatable, and the proximal end of the eighty-second holding support part 2546 is pivotally supported on the proximal end side of the center of the eighty-second holding operation part 2544 so that these components are relatively rotatable. The distal end of the eighty-first holding support part 2526 and the distal end of the eighty-second holding support part 2546 are pivotally supported to be relatively rotatable and movable in the longitudinal direction of the long hole 2555.

Subsequently, operation to open the sheet 910 by the eighth sheet applying system 80 will be described below. Hereinafter, in FIG. 23, the first shaft 901 and the second shaft 902 are rotatably held at the first holding end part 520 and the second holding end part 540, respective, and two sides of the sheet 910 are wound around the first shaft 901 and the second shaft 902, respectively. In FIG. 23, the sheet 910 is opened.

When the operator inserts fingers into the finger holes of the first annular part 311 and the second annular part 321 and then grips the first annular part 311 and the second annular part 321, in other words, shortens the interval between the fingers inserted into the finger holes, the first operation lever 310 rotates relative to the second operation lever 320 and the leading end of the first operation bar part 312 approaches the leading end of the second operation bar part 322. Then, the ball-shaped proximal end 223a moves in the bottomed cylinder hole 313, and the swing movement part 223 is moved by pressing toward the distal end side. Accordingly, the swing movement part 223 presses the first swing-support part 221a and the second swing-support part 221b toward the distal end side through the first swing operation part 222a and the second swing operation part 222b. As described above, the proximal end of the operation cover 323 is fixed to the leading end of the second operation bar part 322, and the distal end thereof pivotally supports the first swing-support part 221a and the second swing-support part 221b. Thus, as the swing movement part 223 presses the first swing operation part 222a and the second swing operation part 222b toward the distal end side, the first swing-support part 221a and the second swing-support part 221b rotate about the pin 224.

Then, the corners of the first swing-support part 221a and the second swing-support part 221b positioned on the distal end side press the first support shaft 210a and the second support shaft 210b toward the distal end side. Accordingly, the first support shaft 210a and the second support shaft 210b move in the longitudinal direction of the first supporter. As described above, the proximal end side of the support cover 211 is pivotally supported together with the distal ends of the first swing-support part 221a and the second swing-support part 221b by the pin 224 so that these components are relatively rotatable, and the distal end side of the support cover 211 is pivotally supported together with the first opening-closing L-shaped part 512 and the second opening-closing L-shaped part 532 by the pin 555 so that these components are relatively rotatable. Thus, as described in the first embodiment, when the operator grips the first annular part 311 and the second annular part 321, the first support shaft 210a and the second support shaft 210b presses the proximal end of the second opening-closing L-shaped part 532 and the proximal end of the first opening-closing L-shaped part 512, and the second opening-closing member 530 and the first opening-closing member 510 rotate about the pin 555 and become separated from the first opening-closing member 510 and the second opening-closing member 530. Accordingly, the first holding end part 520 and the second holding end part 540 are separated from each other. Consequently, the first shaft 901 and the second shaft 902 being rotatably held at the first holding end part 520 and the second holding end part 540, respectively, are separated in parallel to each other, and as a result, the sheet 910 being wound around the first shaft 901 and the second shaft 902 is expanded or unfolded.

As described above, the first holding end part 520 and the second holding end part 540 are connected with the pin 555 through the eighty-first holding operation part 2524, the eighty-second holding operation part 2544, and the holding-movement part 2554, and the distal end of the eighty-first holding support part 2526 and the distal end of the eighty-second holding support part 2546 are pivotally supported to be movable in the longitudinal direction of the long hole 2555. Accordingly, the angle between the eighty-first holding operation part 2524 and the holding-movement part 2554 and the angle between the eighty-second holding operation part 2544 and the holding-movement part 2554 are constantly equal to each other irrespective of opening-closing degrees of the first holding end part 520 and the second holding end part 540, and the first holding end part 520 and the second holding end part 540 are reliably separated in parallel to each other.

Operation to swing or oscillate the eighty-first holder 2500 and a sheet applying method of applying the sheet 910 to an affected part in the body cavity by the eighth sheet applying system 80 are same as those in the first embodiment, and thus description thereof is omitted.

According to the present embodiment, effects same as those of the first to fourth embodiments are achieved. In addition, the first holding end part 520 and the second holding end part 540 can be reliably separated in parallel to each other, and the sheet 910 can be easily applied to the target.

Note that, in any embodiment, the first shaft 901 and the second shaft 902 are rotatable in the connection hole 541, but may be fixed to prevent falling.

Note that, in any embodiment, the proximal ends 223a and 423a may not have ball shapes, and the bottomed cylinder hole 313 may be a hole having a rectangular section in place of a cylinder hole. These components only need to have shapes with which the proximal end 223a is freely movable in the bottomed cylinder hole 313 in the direction in which the bottomed cylinder hole 313 extends.

In any embodiment, the sheet may be self-expandable and may not have a rectangular shape but may have a circular shape, an elliptical shape, an oval shape, a rhombus shape, a square shape, or any other shape that a film can have.

In any embodiment, the cover 920 may not completely cover the sheet 910 but only needs to cover at least part of the sheet 910.

In any embodiment, the first holding end part 520 and the second holding end part 540 may not be separated in parallel to each other but may be separated, for example, to draw a locus of a fan shape. Accordingly, the sheet 910 can be unfolded a fan shape. The length of each member included in the first holder 500 and the second holder 1500 may be adjusted to unfold the first holding end part 520 and the second holding end part 540 in various aspects.

In any embodiment, the first and second holding end parts may not be separated in parallel to each other but may be separated so that the distal ends thereof draw an arc, in other words, locus thereof draw a fan shape.

In any embodiment, the first shaft 901 and the second shaft 902 may be part of a sheet applying device in place of the sheet cartridge. In this case, similarly to any embodiment, the first shaft 901 and the second shaft 902 are rotatably attached to the connection hole 541, and only the sheet 910 is mounted on the sheet applying device. In the sheet applying device including the first shaft 901 and the second shaft 902, the sheet cartridge 900 may be mounted on the sheet applying device after the first shaft 901 and the second shaft 902 are removed.

Although the procedure of causing part of the sheet 910 to contact the target 2 after the sheet 910 is expanded to some extent is described in each embodiment, part of the sheet 910 may be made contact the target 2 before the sheet 910 is expanded or before the sheet 910 is expanded to some extent, and thereafter, the sheet 910 may be expanded.

In any embodiment, when part of the sheet 910 is made contact with the target, a substantially central part or other parts of the sheet 910 may be made contact with the target. A site that is made contact with the target in the sheet 910 is selected as appropriate in accordance with, for example, the position, shape, and state of the target.

Note that the first shaft 901 and the second shaft 902 may be made of any material as long as the sheet can be wound, expanded, unfolded, and applied.

Note that the size and shape of each member indicated in the present specification and the drawings and the number thereof are exemplary, and the member is not limited to the indicated size, shape, and number. In addition, the material of each member is exemplary, and the member is not limited to the material.

The embodiments of the present invention are described above with reference to the accompanying drawings, but it is clear to the skilled person in the art that the structure and relation of each component may be modified without departing from the recited scope and gist of the invention.

1. A sheet applying device comprising:
a supporter configured to be inserted into a tube provided through a body wall; and
a holder configured to be provided at a leading end of the supporter and capable of holding a sheet, and to deform to eject the sheet while expanding the sheet, after the holder holding the sheet protrudes into a body cavity through a leading end of the tube.

2. The sheet applying device according to claim 1, wherein the holder can apply the sheet to a target while expanding the sheet.

3. The sheet applying device according to claim 2, wherein the holder can apply the sheet while expanding the sheet after contacting a substantially central part of the sheet to a target.

4. The sheet applying device according to claim 1, wherein
the holder has such a thin plate shape that the holder can be wound around the holder, is pressed in an expanding direction when being wound, and holds the sheet being wound and enclosed on an inner periphery side of the holder, and
after the holder holding the sheet protrudes into the body cavity through the leading end of the tube, the holder expands the sheet and applies the sheet to a target.

5. The sheet applying device according to claim 1, wherein
the supporter includes
a support shaft extending and movable in a longitudinal direction of the supporter, and
a handle configured to move the support shaft,
the holder includes
a first opening-closing member and a second opening-closing member that are attached to a distal end of the support shaft, and
a first holding end part and a second holding end part that are attached to distal ends of the first and second opening-closing members, respectively, and
when the support shaft is moved toward a distal end by the handle, the first and second opening-closing members are separated from each other due to pressing by the support shaft and the first and second holding end parts are separated from each other.

6. The sheet applying device according to claim 5, wherein
the support shaft includes a first support shaft and a second support shaft extending in parallel to each other,
the handle is attached to proximal ends of the first and second support shafts and moves the first support shaft and/or the second support shaft in the longitudinal direction of the supporter,
the first and second opening-closing members are attached to distal ends of the first and second support shafts, respectively, and
when the first and second support shafts are moved toward the distal ends, the first and second opening-closing members are separated from each other due to pressing by the first and second support shafts and the first and second holding end parts are separated from each other.

7. The sheet applying device according to claim 5, further comprising:
a holding-movement part attached to proximal ends of the first and second opening-closing members;
a first holding operation part rotatably attached to the first opening-closing member;
a second holding operation part rotatably attached to the second opening-closing member;
a first holding support part rotatably attached to the first holding operation part; and
a second holding support part rotatably attached to the second holding operation part, wherein
the first holding operation part has a distal end attached to the first opening-closing member and has a proximal end attached to the holding-movement part,
the second holding operation part has a distal end attached to the second opening-closing member and has a proximal end attached to the holding-movement part,
the holding-movement part includes a long hole on a distal end side,
the proximal ends of the first and second holding operation parts are rotatably attached to the holding-movement part, and
the distal ends of the first and second holding support parts are movably attached in the long hole.

8. The sheet applying device according to claim 5, wherein the first and second holding end parts can be separated in parallel to each other.

9. The sheet applying device according to claim 6, wherein
the handle moves the first support shaft in the longitudinal direction of the supporter, and
when the first support shaft is moved toward the distal end, the first opening-closing member is pressed by the first support shaft and the first and second opening-closing members swing in a plane including the first and second support shafts.

10. The sheet applying device according to claim 1, wherein
the supporter includes a cylindrical part and a pusher stored at a distal end in the cylindrical part, and
the pusher can protrude and expand from the cylindrical part to eject the sheet while expanding the sheet.

11. The sheet applying device according to claim 1, wherein
the supporter includes a bar part,
the holder includes a bending part provided at a leading end of the bar part, and
the bending part can bend to eject the sheet while expanding the sheet.

12. The sheet applying device according to claim 1, wherein the opening-closing member includes a connection part connectable with a surgical operation support robot.

13. A sheet cartridge used for the sheet applying device according to claim 1, the sheet cartridge comprising:
a first shaft and a second shaft having bar shapes;
a sheet including a first part and a second part, the first part being wound around the first shaft, the second part being different from the first part and wound around the second shaft; and
a cover attached to cover at least part of an outer surface of the sheet wound around the first and second shafts.

14. The sheet cartridge according to claim 13, wherein the cover includes a string extending from a proximal end of the cover.

15. A sheet applying system comprising
a sheet applying device
and a sheet cartridge used for the sheet applying device, wherein the sheet applying device comprising:
a supporter configured to be inserted into a tube provided through a body wall; and
a holder configured to be provided at a leading end of the supporter and capable of holding a sheet, and to deform to eject the sheet while expanding the sheet, after the holder holding the sheet protrudes into a body cavity through a leading end of the tube; and
wherein the sheet cartridge comprising:
a first shaft and a second shaft having bar shapes;
a sheet including a first part and a second part, the first part being wound around the first shaft, the second part being different from the first part and would around the second shaft; and
a cover attached to cover at least part of an outer surface of the sheet wound around the first and second shafts.

16. A sheet applying method of using the sheet applying device according to claim 1, the sheet applying method comprising:
a step of protruding at least part of the sheet into the body cavity from the leading end of the tube through inside of the tube;
a step of expanding at least part of the sheet;
a step of causing at least part of the sheet to contact a target; and
a step of entirely expanding the sheet so that the sheet comes off the first and second shafts.

17. The sheet applying method according to claim 16, wherein the coming-off step includes a step of applying the sheet to a target.

18. The sheet applying method according to claim 16, wherein
the contacting step includes a step of causing a substantially central part of the sheet to contact a target, and
the coming-off step is executed after the contacting step and includes a step of applying the sheet to a target.

19. The sheet applying method according to claim 16, wherein the steps are executed
in an order of the protruding step, the expanding step, the contacting step, the coming-off step, and the applying step, or
in an order of the protruding step, the contacting step, the expanding step, the coming-off step, and the applying step.

20. A sheet applying method of using the sheet applying system according to claim 15, the sheet applying method comprising:
a step of protruding at least part of the sheet into the body cavity from the leading end of the tube through inside of the tube;
a step of expanding at least part of the sheet;
a step of causing at least part of the sheet to contact a target; and
a step of entirely expanding the sheet so that the sheet comes off the first and second shafts.

21. The sheet applying method according to claim 20, wherein the coming-off step includes a step of applying the sheet to a target.

22. The sheet applying method according to claim 20, wherein
the contacting step includes a step of causing a substantially central part of the sheet to contact a target, and
the coming-off step is executed after the contacting step and includes a step of applying the sheet to a target.

23. The sheet applying method according to claim 20, wherein the steps are executed
in an order of the protruding step, the expanding step, the contacting step, the coming-off step, and the applying step, or
in an order of the protruding step, the contacting step, the expanding step, the coming-off step, and the applying step.

24. The sheet applying method according to claim 20, wherein
the cover includes a string extending from a proximal end of the cover, and
the sheet applying method further includes, before the step of expanding at least part of the sheet, a step of removing the cover from the sheet by pulling the string to a proximal end side.
